(19) United States
(12) Patent Application Publication
(10) Pub. No.: US 2023/0277536 A1
(43) Pub. Date: Sep. 7, 2023

(54) METHODS AND COMPOSITIONS FOR TREATING EWING FAMILY OF TUMORS

(71) Applicant: Dagang GUO, Beijing (CN)

(72) Inventor: Dagang GUO, Beijing (CN)

(21) Appl. No.: 17/988,386

(22) Filed: Nov. 16, 2022

(30) Foreign Application Priority Data
Nov. 25, 2021 (WO) PCT/CN2021/133165

(51) Int. Cl. A61K 31/506; C12Q 1/6886; A61P 35/00

(52) U.S. Cl. CPC A61K 31/506; C12Q 1/6886; A61P 35/00; G01N 2800/7028

(57) ABSTRACT

Provided is the use of YM155 monobromide for treating the Ewing family of tumors such as Ewing's sarcoma, and related kits, compositions, and methods, including diagnostic methods

### CROSS-REFERENCE TO RELATED APPLICATIONS

This application claims priority to PCT/CN2021/133165, filed Nov. 25, 2021, which is incorporated by reference in its entirety.

### BACKGROUND

### Technical Field

Embodiments of the present disclosure relate to the use of YM155 monobromide for treating the Ewing family of tumors such as Ewing's sarcoma, and related kits, compositions, and methods, including diagnostic methods.

### Description of the Related Art

YM155 monobromide is a small-molecule that exhibits potent antitumor activity (see, e.g., Minematsu et al., Drug Metabolism and Disposition, 37:619-628, 2008). YM-155 exerts anti-tumor effects in various in vivo cancer models, including prostate, pancreatic, and lung cancer (see, e.g., Nakahara et al., Cancer Research 67:8014-8021, 2007; and Na et al., PLoS One 7(6), 2012).

However, there is a need in the art to better predict the anti-cancer therapeutic efficacy of YM155 monobromide, and thereby identify patients that will benefit most from treatment with this chemotherapeutic, and others.

### BRIEF SUMMARY

Embodiments of the present disclosure include methods for treating a Ewing tumor in a human subject in need thereof, comprising administering YM155 monobromide [1−(2-Methoxy ethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, to the subject, thereby treating the Ewing tumor in the subject in need thereof.

In some embodiments, the Ewing tumor is characterized by a chromosomal translocation. In some embodiments, the chromosomal translocation is a reciprocal translocation of the Ewing's Sarcoma Breakpoint Region 1 (EWSR1) gene of chromosome 22 to the Friend Leukemia Virus Integration 1 (FLIT) gene of chromosome 11 (the EWSR1-FLI1 translocation).

Certain embodiments comprise the steps of:
(a) determining if the subject has a Ewing tumor; and
(b) administering the YM155 monobromide, or an analog, derivative, or pharmaceutically acceptable salt thereof, to the subject if the subject has a Ewing tumor.

In some embodiments:
(a) comprises determining if a chromosomal translocation is present in a sample of cancer tissue from the subject; and
(b) comprises administering the YM155 monobromide, or an analog, derivative, or pharmaceutically acceptable salt thereof, to the subject if the chromosomal translocation is present in the sample of cancer tissue.

In some embodiments, the chromosomal translocation is the EWSR1-FLI1 translocation.

Certain embodiments comprise determining if the chromosomal translocation is present in the sample of cancer tissue by performing a diagnostic assay selected from reverse transcription-polymerase chain reaction (RT-PCR) and in situ hybridization (ISH), optionally fluorescence in situ hybridization (FISH).

In particular embodiments:
(a) comprises histological analysis of a sample of cancer tissue from the subject; and
(b) comprises administering the YM155 monobromide, or an analog, derivative, or pharmaceutically acceptable salt thereof, to the subject if the histological analysis identifies any one or more of a small-blue-round-cell tumor by Hematoxylin and eosin (H&E) staining, positive periodic acid—Schiff (PAS) staining, negative Periodic acid—Schiff—diastase (PAS diastase) staining, and/or histological staining of any one of CD99, CD117, CD56, and/or Ki67.

Certain embodiments comprise the step of obtaining the sample of cancer tissue from the subject. In some embodiments, the sample of cancer tissue is a surgical sample, or other biopsy sample obtained from the subject, optionally a biopsy of bone tissue of soft tissue.

In some embodiments, the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject. In some embodiments, the Ewing tumor is metastatic.

Certain embodiments comprise administering the YM155 in combination with at least one additional anti-cancer treatment. In some embodiments, the at least one anti-cancer treatment is selected from one or more of chemotherapy (optionally high dose chemotherapy with stem cell rescue), radiation therapy, surgery, monoclonal antibody therapy, kinase inhibitor therapy, NEDD8-activating enzyme (NAE) inhibitor therapy, and immunotherapy, optionally immune checkpoint inhibitor therapy or CAR T-cell therapy.

Some embodiments include the use of a diagnostic kit for determining therapeutic response to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, therapy in a human subject having or suspected of having a Ewing tumor, comprising means for determining if a chromosomal translocation is present in a sample of cancer tissue from the human subject.

In some embodiments, the chromosomal translocation is the EWSR1-FLI1 translocation. In some embodiments, the means comprise reagents for performing a diagnostic assay selected from reverse transcription-polymerase chain reaction (RT-PCR) and in situ hybridization (ISH), optionally fluorescence in situ hybridization (FISH). In some embodiments, the sample of cancer tissue is a surgical sample, or other biopsy sample obtained from the subject, optionally a biopsy of bone tissue of soft tissue. In some embodiments, the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject. In some embodiments, the diagnostic kit comprises YM155 monobromide, or an analog, derivative, or pharmaceutically acceptable salt thereof.

Some embodiments include a patient care kit, comprising:
(a) means for determining if a chromosomal translocation is present in a sample of cancer tissue from a human subject having or suspected of having a Ewing tumor; and
(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof.

In some embodiments, the chromosomal translocation is the EWSR1-FLI1 translocation. In some embodiments, the means comprise reagents for performing a diagnostic assay selected from reverse transcription-polymerase chain reaction (RT-PCR) and in situ hybridization (ISH), optionally fluorescence in situ hybridization (FISH). In some embodiments, the sample of cancer tissue is a surgical sample, or other biopsy sample obtained from the subject, optionally a biopsy of bone tissue of soft tissue. In some embodiments, the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject.

Certain embodiments relate to a pharmaceutical composition for use in a method of treating a Ewing tumor in a human subject in need thereof, comprising YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof.

In some embodiments, the Ewing tumor is characterized by a chromosomal translocation. In some embodiments, the chromosomal translocation is a reciprocal translocation of the Ewing's Sarcoma Breakpoint Region 1 (EWSR1) gene of chromosome 22 to the Friend Leukemia Virus Integration 1 (FLI1) gene of chromosome 11 (the EWSR1-FLI1 translocation). In some embodiments, the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject. In some embodiments, the Ewing tumor is metastatic.

Certain pharmaceutical compositions are for use in combination with at least one additional anti-cancer treatment. In some embodiments, the at least one anti-cancer treatment is selected from one or more of chemotherapy (optionally high dose chemotherapy with stem cell rescue), radiation therapy, surgery, monoclonal antibody therapy, kinase inhibitor therapy, NEDD8-activating enzyme (NAE) inhibitor therapy, and immunotherapy, optionally immune checkpoint inhibitor therapy or CAR T-cell therapy.

Particular embodiments relate to the use of a composition in the preparation of a medicament for treating a Ewing tumor in a human subject in need thereof, comprising YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof.

In some embodiments, the Ewing tumor is characterized by a chromosomal translocation. In some embodiments, the chromosomal translocation is a reciprocal translocation of the Ewing's Sarcoma Breakpoint Region 1 (EWSR1) gene of chromosome 22 to the Friend Leukemia Virus Integration 1 (FLIT) gene of chromosome 11 (the EWSR1-FLI1 translocation). In some embodiments, the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject. In some embodiments, the Ewing tumor is metastatic.

Certain embodiments are for use in combination with at least one additional anti-cancer treatment. In some embodiments, the at least one anti-cancer treatment is selected from one or more of chemotherapy (optionally high dose chemotherapy with stem cell rescue), radiation therapy, surgery, monoclonal antibody therapy, kinase inhibitor therapy, NEDD8-activating enzyme (NAE) inhibitor therapy, and immunotherapy, optionally immune checkpoint inhibitor therapy or CAR T-cell therapy.

### BRIEF DESCRIPTION OF THE FIGURES

FIGS. 1A-1B show the YM155 dose response curves and ICso determinations for Ewing tumors cell lines A673 (1A) and SK-N-MC (2B).

FIGS. 2A-2D show the results of apoptosis assays after treating A673 cells with DMSO (control) or YM155 (10 nM). Annexin V assay (2A-2B); caspase 3 activity assay (2C-2D).

FIGS. 3A-3D show the results of apoptosis assays after treating SK-N-MC cells with DMSO (control) or YM155 (10 nM). Annexin V assay (3A-3B); caspase 3 activity assay (3C-3D).

FIGS. 4A-4C shows the results of YM155 treatment in murine xenograft Ewing tumor models. FIGS. 4A-4B show the murine A673 xenograft model. The dosing schedules in FIG. 4B are as follows: YM155 monobromide, 3.5 mg/kg, 21-day-infusion with an osmotic pump; vincristine, 0.5 mg/kg, QW*3, IV; doxorubicin, 3.3 mg/kg, QW*3, IV; cyclophosphamide, 40 mg/kg, QW*3, IV. The dosing schedules in FIG. 4B are as follows: YM155 monobromide, 2 mg/kg, 21-day-infusion with an osmotic pump; ifosfamide, 30 mg/kg, QW*3, IV; etoposide, 7.5 mg/kg, QW*3, IP. FIG. 4C shows the murine SK-N-MC xenograft model. The dosing schedules were as follows: YM155 monobromide, 3.5 mg/kg, 21-day-infusion with an osmotic pump; vincristine, 0.5 mg/kg, QW*3, IV; doxorubicin, 3.3 mg/kg, QW*3, IV; cyclophosphamide, 40 mg/kg, QW*3, IV.

FIG. 5 shows a Western-blot for EWS-FLI1, MYC, and cyclin D1 expression level in A673 and SK-N-MC cells following treatment with 10 nM of YM155 (PC002).

### DETAILED DESCRIPTION

Embodiments of the present disclosure relate to the surprising discovery that the Ewing family of tumors (for example, Ewing's sarcoma) show significant sensitivity to YM155 monobromide therapy, particular such tumors with the EWSR1-FLI1 translocation. Thus, the EWSR1-FLI1 translocation and other phenotypes of a the Ewing family of tumors can be used as biomarkers or companion diagnostics to optimize YM155-related cancer therapies.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

An “antagonist” or “inhibitor” refers to biological structure or chemical agent that interferes with or otherwise reduces the physiological action of another molecule, such as a protein. In some instances, the antagonist or inhibitor specifically binds to the other molecule and/or a functional ligand of the other molecule. In some instances, the antagonist or inhibitor down-regulates the expression of the other molecule. Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

By “about” is meant a quantity, level, value, number, frequency, percentage, dimension, size, amount, weight or length that varies by as much as 20, 15, 10, 9, 8, 7, 6, 5, 4, 3, 2, or 1% to a reference quantity, level, value, number, frequency, percentage, dimension, size, amount, weight, or length.

The term “binding” refers to a direct association between two molecules, due to, for example, covalent, electrostatic, hydrophobic, and ionic and/or hydrogen-bond interactions, including interactions such as salt bridges and water bridges.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “half maximal effective concentration” or “EC50” refers to the concentration of an agent as described herein at which it induces a response halfway between the baseline and maximum after some specified exposure time; the EC50 of a graded dose response curve therefore represents the concentration of a compound at which 50% of its maximal effect is observed. EC50 also represents the plasma concentration required for obtaining 50% of a maximum effect in vivo Similarly, the “EC90” refers to the concentration of an agent or composition at which 90% of its maximal effect is observed. The “EC90” can be calculated from the “EC50” and the Hill slope, or it can be determined from the data directly, using routine knowledge in the art. In some embodiments, the EC50 of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC50 value of about 1 nM or less.

The “half maximal inhibitory concentration” (or “IC50”) is a measure of the potency of an agent in inhibiting a specific biological or biochemical function. This quantitative measure indicates how much of a particular agent (inhibitor) is needed to inhibit a given biological process (or component of a process, i.e. an enzyme, cell, cell receptor or microorganism) by half. The values are typically expressed as molar concentration. The concentration is commonly used as a measure of antagonist drug potency in pharmacological research. In some instances, IC50 represents the concentration of an agent that is required for 50% inhibition in vitro. The IC50 of an agent can be determined by constructing a dose-response curve and examining the effect of different concentrations of the agent on the desired activity, for example, inhibition of tumor cell proliferation, tumor-cell killing.

The “half-life” of an agent refers to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000-fold or more of the amount produced by no composition or a control composition (e.g., the absence of agent or a different agent). An “increased,” “stimulated” or “enhanced” amount may also include an amount that is about or at least about 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, 2000%, 3000%, 4000%, 5000% or more of the amount produced by no composition or a control composition. A “decreased” or “reduced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, or 5000-fold less of the amount produced by no composition or a control composition. A “decreased” or “reduced” amount may also include a 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000%, 2000%, 3000%, 4000%, or 5000% less of the amount produced by no composition or a control composition. Examples of comparisons and “statistically significant” amounts are described herein.

“Prodrug” is meant to indicate a compound that may be converted under physiological conditions or by solvolysis to a biologically active compound described herein, for example, a GSI compound. Thus, the term “prodrug” refers to a metabolic precursor of a compound that is pharmaceutically acceptable. A prodrug may be inactive when administered to a subject in need thereof, but is converted in vivo to an active compound. Prodrugs may be rapidly transformed in vivo to yield the parent compound, for example, by hydrolysis in blood. The prodrug compound often offers advantages of solubility, tissue compatibility or delayed release in a mammalian organism (see, for example, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987. Examples of prodrugs include, but are not limited to, acetate, formate, and benzoate derivatives of alcohol or amide derivatives of amine functional groups in the compounds of the disclosure and the like.

The term “prodrug” is also meant to include any covalently bonded carriers, which release the active compound in vivo when such prodrug is administered to a subject. Prodrugs of a compound may be prepared by modifying functional groups present in the compound in such a way that the modifications are cleaved, either in routine manipulation or in vivo, to the parent compound. Prodrugs include compounds where a hydroxy, amino, or mercapto group is bonded to any group that, when the prodrug of the compound is administered to a subject, cleaves to form a free hydroxy, free amino, or free mercapto group, respectively.

“Pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, for example, which has been approved by the United States Food and Drug Administration as being acceptable for use in humans or domestic animals.

“Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, the term “solvate” refers to an aggregate that comprises one or more molecules of a compound described herein with one or more molecules of solvent. The solvent may be water, in which case the solvate may be a hydrate. Alternatively, the solvent may be a biologically-inert organic solvent. Thus, the compounds described herein may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound may merely retain adventitious water or be a mixture of water plus some adventitious solvent.

A “pharmaceutical composition” refers to a formulation of a YM155 compound described herein and a medium generally accepted in the art for the delivery of the biologically active compound to mammals, e.g., humans. Such a medium includes all pharmaceutically acceptable carriers, diluents, and excipients.

The YM155 compounds described herein, or their pharmaceutically-acceptable salts, may contain one or more asymmetric centers and may thus give rise to enantiomers, diastereomers, and other stereoisomeric forms that may be defined, in terms of absolute stereochemistry, as (R)- or (5)- or, as (D)- or (L)- for amino acids. The present disclosure is meant to include all such possible isomers, as well as their racemic and optically pure forms. Optically active (+) and (−), (R)- and (5)—, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

In certain embodiments, the “purity” of any given agent in a composition may be defined. For instance, certain compositions may comprise an agent that is at least 80%, 85%, 90%, 91%, 92%, 93%, 94%, 95%, 96%, 97%, 98%, 99%, or 100% pure on a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO4). In specific embodiments, solubility is measured at relatively lower pH (e.g., pH 6.0) and relatively higher salt (e.g., 500 mM NaCl and 10 mM NaPO4). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

“Stable compound” and “stable structure” are meant to indicate a compound that is sufficiently robust to survive isolation to a useful degree of purity from a reaction mixture, and formulation into a therapeutic agent.

The term “polynucleotide” and “nucleic acid” includes mRNA, RNA, cRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given reference quantity, level, value, number, frequency, percentage, dimension, size, amount, weight, length, or other.

A “stereoisomer” refers to a compound made up of the same atoms bonded by the same bonds but having different three-dimensional structures, which are not interchangeable. The present disclosure includes various stereoisomers and mixtures thereof and includes “enantiomers”, which refers to two stereoisomers whose molecules are non-superimposeable mirror images of one another.

A “tautomer” refers to a proton shift from one atom of a molecule to another atom of the same molecule. The present disclosure includes tautomers of any said compounds.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Embodiments of the present disclosure include methods for treating a cancer comprising a “Ewing family of tumors”, referred to herein as a “Ewing tumor”, in a human subject in need thereof. The “Ewing family of tumors” or “Ewing tumor(s)” for short, originally characterized as “Ewing's sarcoma” (Ewing, Clin Orthop Relat Res. 450: 25-7, 2006), refers to a group of cancers that includes Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall) (see, for example, Iwamoto, Jpn. J. Clin. Oncol. 37: 79-89, 2007).

Certain embodiments thus include methods of treating a Ewing tumor (for example, Ewing's sarcoma) in a human subject in need thereof, administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, to the subject, thereby treating the Ewing tumor in the subject in need thereof. In certain embodiments, the Ewing tumor is an ETB, an EOE tumor, a PNET, or an Askin tumor, for example, a bone sarcoma or a soft tissue sarcoma, for example, in the legs, pelvis, and/or chest wall of the subject. In specific embodiments, the Ewing tumor is metastatic.

In some instances, the Ewing tumor is the result of a reciprocal translocation between chromosomes 11 and 22, t(11,22), which fuses the Ewing's Sarcoma Breakpoint Region 1(EWSR1) gene of chromosome 22 (which encodes the EWS protein) to the Friend Leukemia Virus Integration 1 (FLIT) gene (which encodes Friend Leukemia Integration 1 transcription factor (FLIT), a member of the ETS transcription factor family) of chromosome 11—that is, the EWSR1-FLI1 translocation.

The EWSR1-FLI1 translocation causes the EWS trans-activation domain (which is usually silent in the wild type) to become highly active, which leads to the translation of a non-natural EWS-FLI1 fusion protein (Riggi et al., New England Journal of Med. 384: 154-164, 2021). The EWS-FLI1 fusion protein has phase transition properties, allowing it to transition into liquid-like, phase separated compartments consisting of membrane-less organelles. These properties allows the fusion protein to access and activate micro-satellite regions of the genome that would otherwise be inaccessible. Here, the fusion protein can convert usually silent chromatin regions into fully active enhancers leading to oncogenesis of the cells. It can also cause variable expression of the genome via epigenetic mechanisms, for example, by recruiting enzymes that affect DNA methylation, histone acetylation, and direct inhibition of non-coding microRNA (Id.). Such leads to increased pluripotency, decreased differentiation of cells, and increased oncogenesis.

Thus, in certain embodiments, the Ewing tumor is characterized by a chromosomal translocation, including a reciprocal translocation. In specific embodiments, the Ewing tumor is characterized by the EWSR1-FLI1 translocation.

Some methods of treating a Ewing tumor include the steps of:
(a) determining if the subject has a Ewing tumor; and
(b) administering the YM155 monobromide, or an analog, derivative, or pharmaceutically acceptable salt thereof, to the subject if the subject has a Ewing tumor.

As noted above, most cases of Ewing tumors are characterized by a chromosomal translocation (e.g., reciprocal translocation, such as the EWSR1-FLI1 translocation). Thus, in certain embodiments, the step of determining if the subject has a Ewing tumor comprises determining if a chromosomal translocation is present in a sample of cancer tissue from the subject. In specific embodiments, the chromosomal translocation is the EWSR1-FLI1 translocation. Such determinations can be made by employing any one or more diagnostic assays in the art (see, for example, Rodriguez and Martin, Methods Mol Biol. 2226: 85-103, 2021; Bridge et al., Mod Pathol. 19: 1-8, 2006). For instance, certain embodiments comprise determining if the chromosomal translocation is present in the sample of cancer tissue by performing a diagnostic assay selected from reverse transcription-polymerase chain reaction (RT-PCR) and in situ hybridization (ISH), including fluorescence in situ hybridization (FISH). RT-PCR) is a laboratory technique combining reverse transcription of RNA into DNA (or cDNA) and amplification of specific DNA targets using polymerase chain reaction (PCR) (see, for example, Freeman et al., BioTechniques. 26: 112-22, 124-5, 1999). In situ hybridization (ISH) and fluorescent in situ hybridization (FISH) refer to a type of hybridization that uses a labeled complementary DNA, RNA or modified nucleic acids strand (i.e., probe) to localize a specific DNA or RNA sequence in a portion or section of tissue (in situ) (see, for example, Parra & Windle, Nature Genetics. 5:17-21, 1993; Gall & Pardue, PNAS USA. 63: 378-383, 1969). Thus, the step of determining if a chromosomal translocation is present in a sample of cancer tissue from the subject can be performed according to routine techniques in the art. In some instances, the methods and kits described herein employ any one or more of the foregoing techniques and/or comprise reagents for performing the same.

Diagnosis of a Ewing tumor can also be based, for example, on histomorphologic and/or immunohistochemical findings. Morphologically, a Ewing tumor is a small-blue-round-cell tumor that typically has a clear cytoplasm on H&E staining, due to glycogen. The presence of glycogen can be demonstrated with positive PAS staining and negative PAS diastase staining Immunohistochemically, a Ewing tumor typically stains positive for CD99, which diffusely marks the cell membrane; however, as CD99 is not necessarily specific for a Ewing tumor, and several auxiliary immunohistochemical markers can be employed to support the histological diagnosis (see, for example, McCuiston et al., Head and Neck Pathol. 12: 89-94, 2018). As noted above, morphologic and immunohistochemical findings can corroborated with an associated chromosomal translocation, such as the EWSR1-FLI1 translocation.

Thus, in particular embodiments, the step of determining if the subject has a Ewing tumor comprises histological analysis of a sample of cancer tissue from the subject; and the methods provided herein comprise administering the YM155 monobromide, or an analog, derivative, or pharmaceutically acceptable salt thereof, to the subject if the histological analysis identifies any one or more of a small-blue-round-cell tumor by Hematoxylin and eosin (H&E) staining, positive periodic acid—Schiff (PAS) staining, negative Periodic acid—Schiff—diastase (PAS diastase) staining, and/or histological staining of any one of CD99, CD117, CD56, and/or Ki67 relative to a reference.

Also included are methods for predicting therapeutic response to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, in a subject having or suspected of having a Ewing tumor, comprising:

(a) determining if a chromosomal translocation is present in a sample of cancer tissue from the human subject, for example, wherein the chromosomal translocation is the EWSR1-FLI1 translocation; and

(b) (i) characterizing the subject as responsive to YM155 monobromide therapy if the chromosomal translocation is present in the sample of cancer tissue; or
(ii) characterizing the subject as non-responsive to YM155 monobromide therapy if the chromosomal translocation is absent in the sample of cancer tissue, thereby predicting therapeutic response to YM155 monobromide in the subject with cancer. Some embodiments include administering YM155 monobromide to the subject if the subject is characterized as responsive to YM155 monobromide therapy. Some instances include administering to the subject a chemotherapeutic agent excluding YM155 monobromide if the subject is characterized as non-responsive to YM155 monobromide therapy.

In certain embodiments, the sample of cancer tissue is a surgical sample, or other biopsy sample obtained from the subject, for example, a biopsy of bone tissue or soft tissue suspected of being cancerous.

Examples of a “reference” include a value, amount, sequence, or other characteristic obtained from a database, for example, a “wild-type” sequence. A “reference” also includes value, amount, sequence, or other characteristic obtained from a non-cancerous tissue from one or more controls, for example, one or more healthy or non-cancerous control subjects (e.g., a population of healthy or non-cancerous control subjects), or one or more corresponding non-cancerous control tissues from the subject being tested. Typically, a “corresponding” non-cancerous control tissue is obtained from the same type of tissue as the cancer tissue being tested. As with the cancer tissue, the reference levels from a non-cancerous control can be determined by any variety of methods, including, for example, by IHC, for example, chromogenic or fluorescent IHC, ELISA, or Western blot on a protein or gene of interest.

In some embodiments, the subject is a human subject. In some embodiments, prior to treatment with YM155, the human subject has received at least 1 or 2 lines of systemic therapy for the Ewing tumor and has relapsed from the last systemic therapy. Examples of prior systemic therapy include chemotherapy, for example, multidrug chemotherapy (e.g., any combination of vincristine, doxorubicin, cyclophosphamide, ifosfamide, and etoposide), surgery, and/or radiation therapy, for example, proton beam radiation therapy.

Certain embodiments include combination therapies, for example, administering YM155 to the subject with a Ewing tumor in combination with at least one or two or more additional chemotherapeutic agents. In some embodiments, the at least one anti-cancer treatment is selected from one or more of chemotherapy (e.g., high dose chemotherapy with stem cell rescue), radiation therapy, surgery, monoclonal antibody therapy, kinase inhibitor therapy, NEDD8-activating enzyme (NAE) inhibitor therapy, and immunotherapy, for instance, immune checkpoint inhibitor therapy or CAR T-cell therapy.

The methods described herein can be used in the treatment and/or diagnosis of any variety of Ewing tumors, including bone and soft tissue sarcomas. In some embodiments, the Ewing tumor is a primary cancer, that is, a cancer growing at the anatomical site where tumor progression began and yielded a cancerous mass. In some embodiments, the Ewing tumor is a secondary or metastatic cancer, that is, a cancer which has spread from the primary site or tissue of origin into one or more different sites or tissues.

In certain embodiments, the methods and compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and compositions described herein increase cancer cell-killing in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, the methods and compositions described herein decrease levels of one or more Ewing tumor markers, such as CD99, CD117, CD56, and/or Ki67, including combinations thereof, by a statistically or clinically-significant amount, for example, by about or at least about 10, 15, 20, 25, 30, 35, 40, 45, 50, 60, 70, 80, 90, 100, 200, 300, 400, 500, 600, 700, 800, 900, 1000, 2000% or more, relative to a control or an earlier timepoint (e.g., before YM155 treatment).

In some embodiments, the methods and compositions described herein increase progression-free survival, overall survival, and/or survival post-progression in the subject in need thereof, for example, by about or at least about 2, 3, 4, 5, 6, 7, 8, 9, 10, 11, 12, 13, 14, 15, 16, 17, 18, 19, 20, 21, 22, 23, or 24 months or more, or by about or at least about 1, 2, 3, 4, 5, 6, 7, 8, 9, or 10 years or more.

In certain embodiments, the methods and compositions described are sufficient to result in stable disease. In certain embodiments, the methods and compositions described herein are sufficient to result in clinically relevant reduction in symptoms of a particular disease indication known to the skilled clinician.

As noted above, the methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, for instance, for the treatment of human disease or testing, the agents described herein are generally incorporated into one or more therapeutic or pharmaceutical compositions prior to administration.

To prepare a therapeutic or pharmaceutical composition, an effective or desired amount of one or more agents is typically mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Certain embodiments include administering a dosage regimen of YM155 via continuous intravenous administration, for example, at about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/m2/day of YM155 by continuous intravenous infusion for about 5, 6, or 7 days. In some instances, the foregoing dosing regimen is repeated every 2, 3, or 4 weeks or so.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Certain embodiments include the use of a diagnostic kit for determining or predicting a therapeutic response (or responsiveness) to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, therapy in a human subject having or suspected of having a Ewing tumor, comprising means for determining if a chromosomal translocation is present in a sample of cancer tissue from the human subject. Also included are patient care kits, comprising: (a) means for determining if a chromosomal translocation is present in a sample of cancer tissue from the human subject; and (b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof. In some embodiments, the chromosomal translocation is the EWSR1-FLI1 translocation.

In particular embodiments, the means for determining if a chromosomal translocation is present in a sample of cancer tissue from the human subject comprise reagents for performing a diagnostic assay selected from reverse transcription-polymerase chain reaction (RT-PCR) and in situ hybridization (ISH), including fluorescence in situ hybridization (FISH). In certain embodiments, the reagents are specific to identifying the EWSR1-FLI1 translocation, and include, for example, RT-PCR primers and/or IHC reagents specific to the EWSR1-FLI1 translocation.

Some diagnostic or patient care kits include a EWSR1 gene reference and/or a FL1 gene reference obtained from a database, or determined from a non-cancerous tissue from a control or reference. The kits can also include written instructions, for example, on how to determine the presence of a chromosomal translocation (for example, the EWSR1-FLI1 translocation) in a tissue sample from a subject.

In some embodiments, a diagnostic or patient care kit contains separate containers, dividers, or compartments for the composition(s) and informational material(s). For example, the composition(s) or reagents can be contained in a bottle, vial, or syringe, and the informational material(s) can be contained in association with the container. In some embodiments, the separate elements of the kit are contained within a single, undivided container. For example, the composition(s) or reagents are contained in a bottle, vial or syringe that has attached thereto the informational material in the form of a label. In some embodiments, the kit includes a plurality (e.g., a pack) of individual containers, each containing one or more compositions, reagents, and/or unit dosage forms of YM155 monobromide. For example, the kit includes a plurality of syringes, ampules, foil packets, or blister packs, each containing a reagent or a single unit dose of YM155 monobromide. The containers of the kits can be air tight, waterproof (e.g., impermeable to changes in moisture or evaporation), and/or light-tight.

The patient care kit optionally includes a device suitable for administration of the agent(s), e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

In certain aspects, the diagnostic or therapeutic response tests or methods described herein are performed at a diagnostic laboratory, and the results are then provided to the subject, or to a physician or other healthcare provider that plays a role in the subject's healthcare and cancer treatment. Particular embodiments thus include methods for providing the results of the responsiveness test to the subject in need thereof, or to the physician or other healthcare provider. These results or data can be in the form of a hard-copy or paper-copy, or an electronic form, such as a computer-readable medium.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

### Activity of YM155 in Ewing Tumor Cell Lines

In vitro cell proliferation assays were performed to test the sensitivity of Ewing tumor cell lines to treatment with YM155. The A673 and SK-N-MC cell lines, each of which have the EWSR1-FLI1 translocation that is characteristic of most cases of Ewing tumors, were seeded in 96-well plates at 2×10³ cells/well and allowed to attach overnight at 37° C. and 5% CO2. The cells were exposed to YM155 at designated concentrations (2-fold serial dilutions starting from 200 nM) for 72 hrs.

Following drug exposure, the cells were labeled with 5-ethynyl-2′- deoxyuridine (EdU) to assess the tumor cell proliferation rates. The labeling lasted 4 hours in the presence of drug exposure. In the control group, the tumor cells received no drug exposure with media change (with 0.1% DMSO), but were similarly labeled with EdU.

The labeled cells were fixed with formaldehyde for 30 min, followed by staining with Hoechst 33342 in a buffer containing 0.5% triton X-100. The incorporated Edu was detected by Click-iT reaction where fixed cells were incubated with a reaction mixture containing 1X Click-iT EdU reaction buffer, CuSO4, and azide-conjugated fluorescent dye in the dark. The stained cells were washed with PBS two times before image acquisition and analysis.

The stained tumor cells were imaged by a high-content screening (HCS) platform (Thermo Scientific Cellomics Array Scan XTi HCS reader). The 10X objective was used to collect images. Twenty-five fields were imaged for each well for the analysis. From the images fluorescent signals for cell nucleus and the EdU incorporated in newly synthesized DNA were obtained from the HCS reader. Inhibition rate=(EdU positive cells in treatment/Edu positive cells in control)*100%. The IC50 was calculated from the dose response curve of drug inhibition.

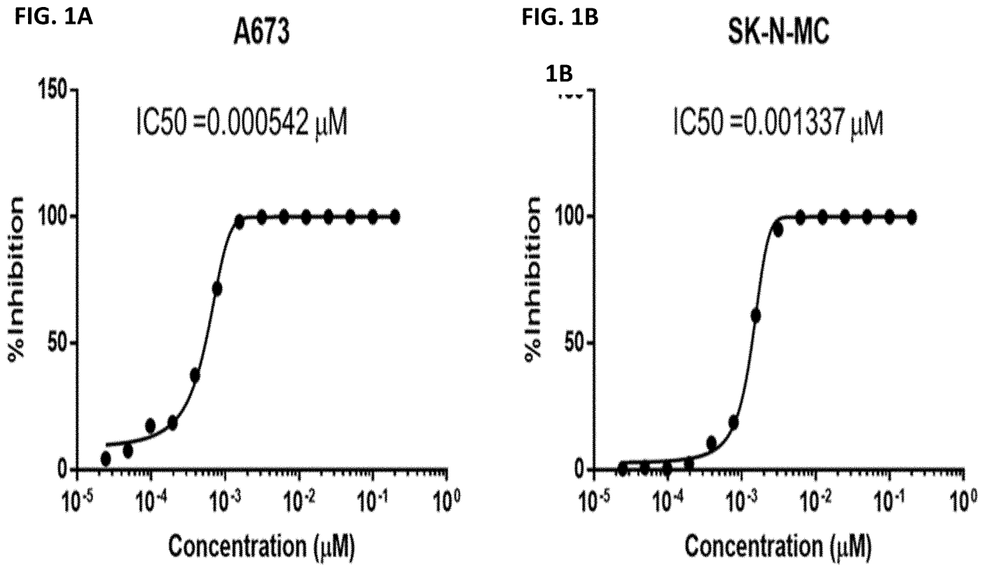

The results are shown in FIGS. 1A-1B, which provide the YM155 dose response curves and IC50 determinations for Ewing tumor cell lines A673 (1A) and SK-N-MC (1B). These results show that two Ewing tumor cell lines with the EWSR1-FLI1 translocation were highly sensitive to YM155 in an in vitro cell proliferation assay.

Cellular apoptosis was then assayed with the Alexa Fluor™ 488 Annexin V/Dead cell apoptosis Kit (ThermoFisher®, USA) and GreenNuc™ live cell caspase 3 activity assay kit (Beyotime Biotechnology, China). Briefly, cells cultured in 6-well tissue culture plates were treated with 10 nM YM155 or DMSO (control). Following 3 days drug exposure, the cells were collected and assayed for apoptosis according to the manufacturers' instructions. For annexin V assay, the cell suspensions were incubated with Alexa Flour™ 488 labeled annexin V and Propidium Iodide (PI). For the caspase 3 activity assay, a caspase 3 substrate was added to generate fluorescent product. The cells were then analyzed on a BD LSR Fortessa cell analyzer. Flow cytometry data was analyzed with Flowjo software.

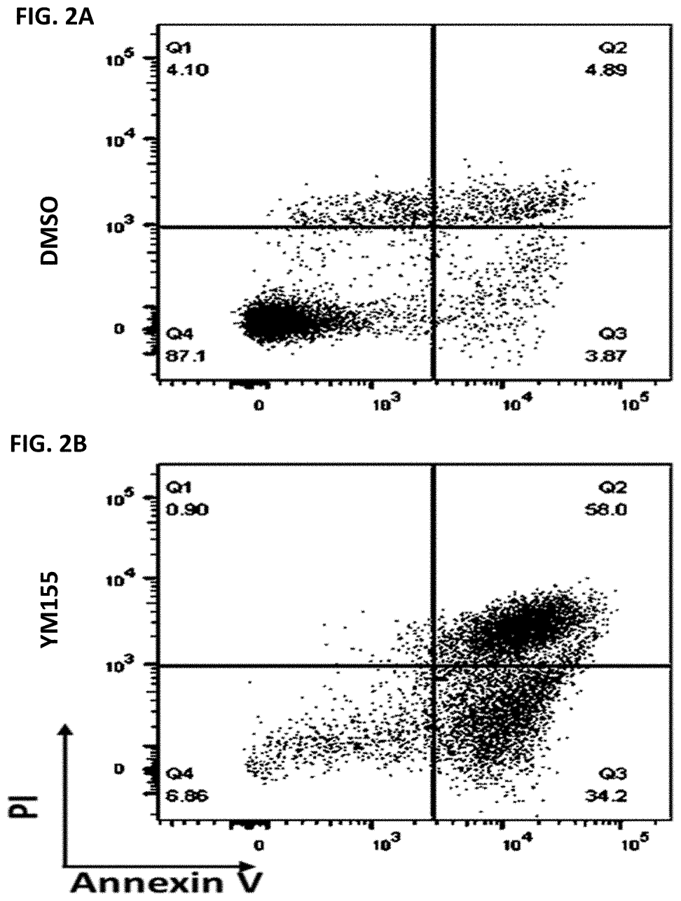

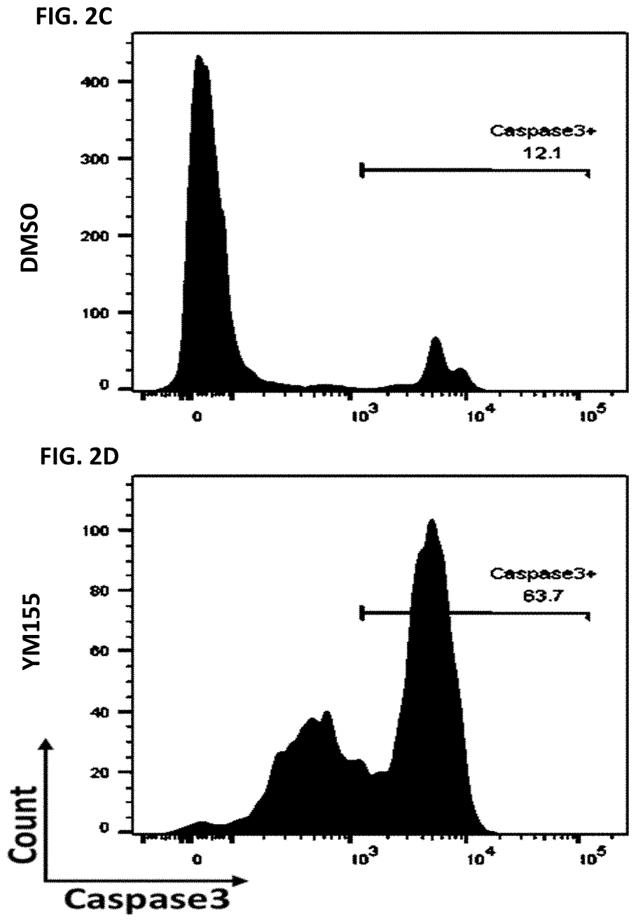

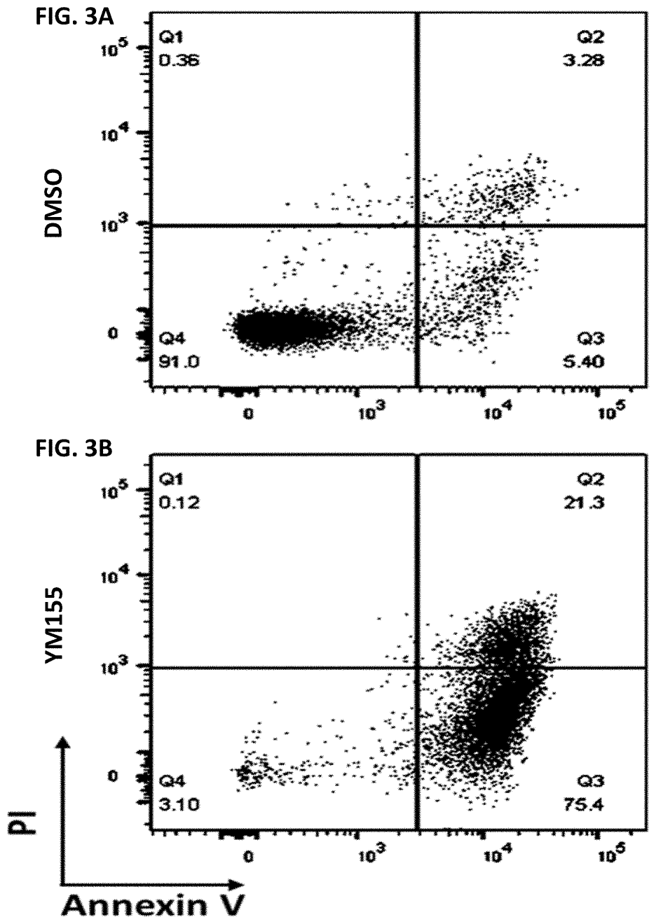

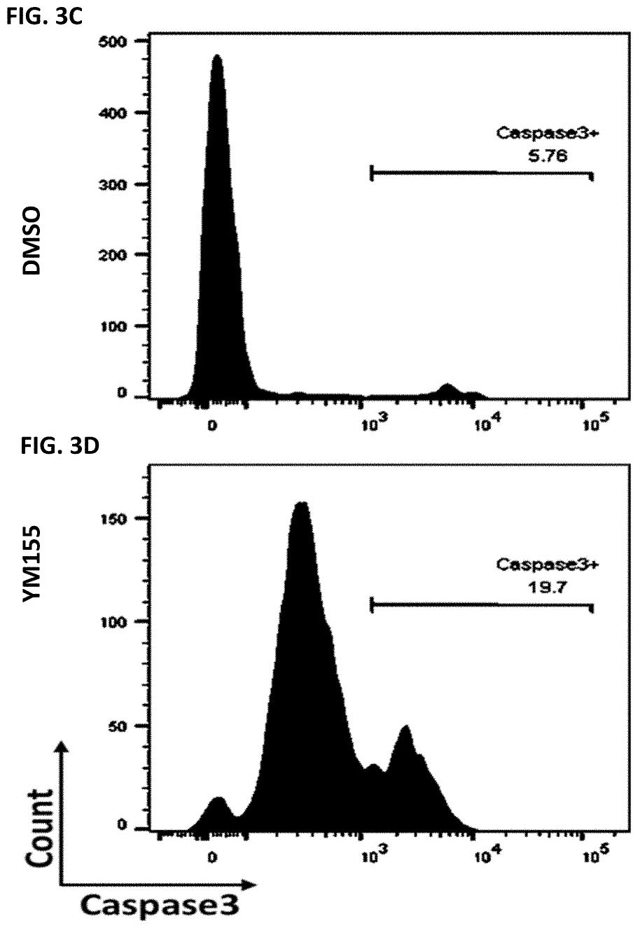

The results are shown in FIGS. 2A-2D (A673) and FIGS. 3A-3D (SK-N-MC), evidencing that YM155 induced strong apoptosis in A673 and SK-N-MC cells.

To test for in vivo activity, a Ewing tumor model was generated by implanting either A673 or SK-N-MC cells into mice. When the tumor size reached 150 mm3, the mice were grouped randomly and injected with YM155 by 21-day infusion with an osmotic pump. The tumor size was measured every three days.

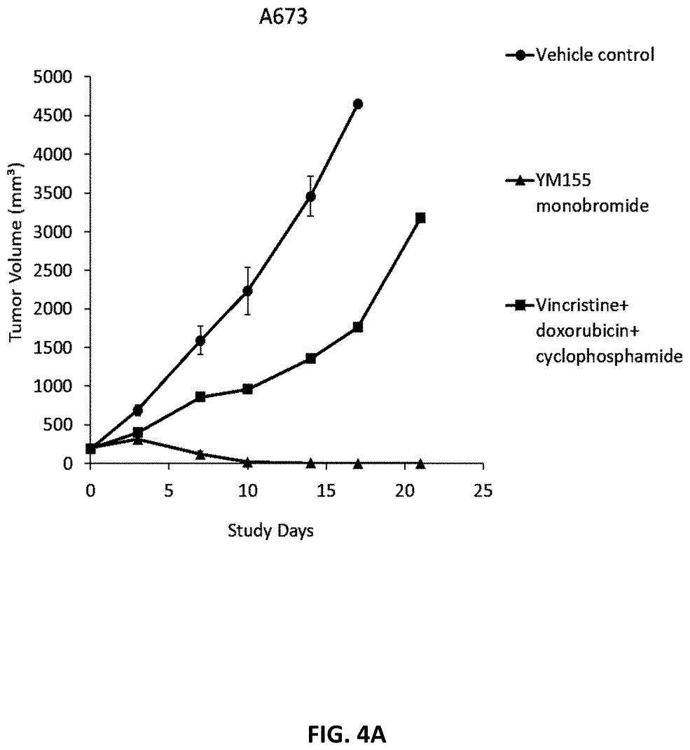

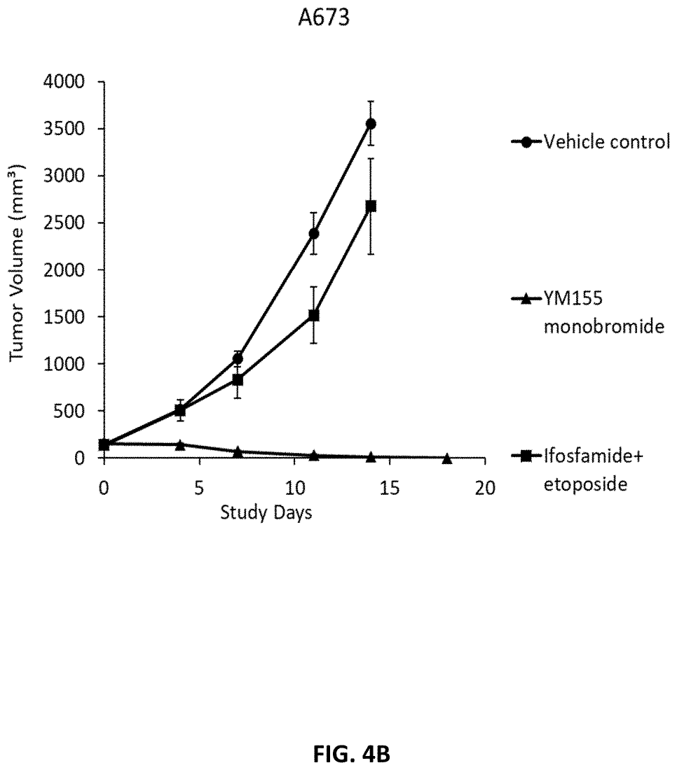

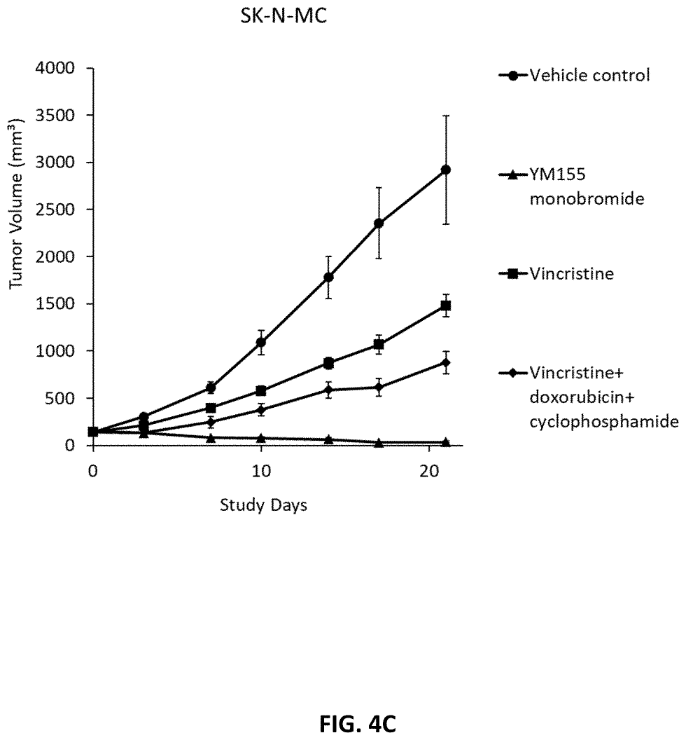

FIGS. 4A-4C show that YM155 had a strong inhibitory effect on tumor growth in each of the A673 and SK-N-MC xenograft models. The efficacy of YM155 was superior to both vincristine standalone therapy, which is used as a first-line chemotherapeutic for treating Ewing tumors, and vincristine/doxorubicin/cyclophosphamide triple therapy. The efficacy of YM155 was also superior to ifosfamide/etoposide combination therapy.

Western-blot analysis was performed to examine the suppression by YM155 on the expression level of EWS-FLI1, the protein product of EWSR1-FLI1 translocation, and its effector proteins MYC and cyclin D1. In brief, A673 and SK-N-MC cells seeded on 6-well plates were treated with 10 nM YM155 for 12-48 hrs. The cells were then harvested and lysed in a lysis buffer containing protease inhibitor cocktail (Beyotime, China) at 4C. The concentrations of total protein in the cell lysates were determined by BCA Protein Assay Kit (ThermoFisher, USA). Forty micrograms of total protein was used in each lane for SDS-PAGE and western-blot assay. Antibodies against EWS-FLI1, MYC, and cyclin D1 were used to detect the expression levels of the proteins. GAPDH was used as a loading control.

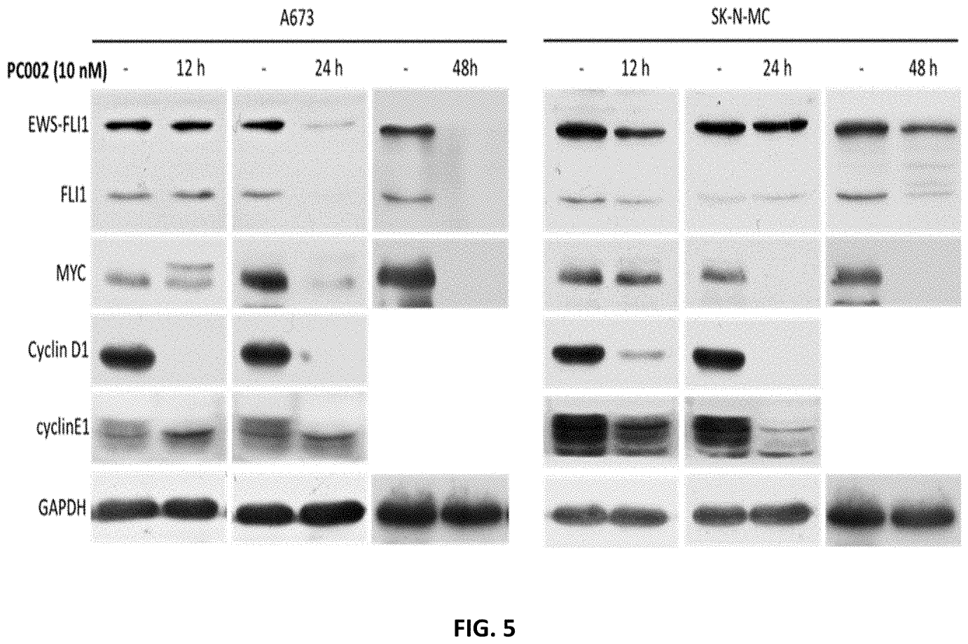

As shown in FIG. 5, YM155 inhibited protein expression of EWS-FLI1, MYC, and cyclin D1 in Ewing tumor cell lines.

Overall, YM155 inhibited EWS-FLI1 and its downstream effectors MYC and cyclin D1. In both in vitro and in vivo experiments, YM155 exhibited potent anti-tumor effect in Ewing tumor models. YM155 is therefore a potential therapeutic drug for treating Ewing tumors with the EWSR1-Fill translocation.

1. A method for treating a Ewing tumor in a human subject in need thereof, comprising
administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, to the subject,
thereby treating the Ewing tumor in the subject in need thereof.

2. The method of claim 1, wherein the Ewing tumor is characterized by a chromosomal translocation.

3. The method of claim 2, wherein the chromosomal translocation is a reciprocal translocation of the Ewing's Sarcoma Breakpoint Region 1 (EWSR1) gene of chromosome 22 to the Friend Leukemia Virus Integration 1 (FLI1) gene of chromosome 11 (the EWSR1-FLIT translocation).

4. The method of claim 1, comprising:
(a) determining if the subject has a Ewing tumor; and
(b) administering the YM155 monobromide, or an analog, derivative, or pharmaceutically acceptable salt thereof, to the subject if the subject has a Ewing tumor.

5. The method of claim 4, wherein:
(a) comprises determining if a chromosomal translocation is present in a sample of cancer tissue from the subject; and
(b) comprises administering the YM155 monobromide, or an analog, derivative, or pharmaceutically acceptable salt thereof, to the subject if the chromosomal translocation is present in the sample of cancer tissue.

6. The method of claim 5, wherein the chromosomal translocation is the EWSR1-FLI1 translocation.

7. The method of claim 5, comprising determining if the chromosomal translocation is present in the sample of cancer tissue by performing a diagnostic assay selected from reverse transcription-polymerase chain reaction (RT-PCR) and in situ hybridization (ISH), optionally fluorescence in situ hybridization (FISH).

8. The method of claim 4, wherein:
(a) comprises histological analysis of a sample of cancer tissue from the subject; and
(b) comprises administering the YM155 monobromide, or an analog, derivative, or pharmaceutically acceptable salt thereof, to the subject if the histological analysis identifies any one or more of a small-blue-round-cell tumor by Hematoxylin and eosin (H&E) staining, positive periodic acid—Schiff (PAS) staining, negative Periodic acid—Schiff—diastase (PAS diastase) staining, and/or histological staining of any one of CD99, CD117, CD56, and/or Ki67.

9. The method of claim 1, comprising obtaining the sample of cancer tissue from the subject.

10. The method of claim 1, wherein the sample of cancer tissue is a surgical sample, or other biopsy sample obtained from the subject, optionally a biopsy of bone tissue of soft tissue.

11. The method of claim 1, wherein the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject.

12. The method of claim 1, wherein the Ewing tumor is metastatic.

13. The method of claim 1, comprising administering the YM155 in combination with at least one additional anti-cancer treatment.

14. The method of claim 13, wherein the at least one anti-cancer treatment is selected from one or more of chemotherapy (optionally high dose chemotherapy with stem cell rescue), radiation therapy, surgery, monoclonal antibody therapy, kinase inhibitor therapy, NEDD8-activating enzyme (NAE) inhibitor therapy, and immunotherapy, optionally immune checkpoint inhibitor therapy or CAR T-cell therapy.

15-20. (canceled)

21. A patient care kit, comprising:
(a) means for determining if a chromosomal translocation is present in a sample of cancer tissue from a human subject having or suspected of having a Ewing tumor; and
(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof.

22. The patient care kit of claim 21, wherein the chromosomal translocation is the EWSR1-FLI1 translocation.

23. The patient care kit of claim 21, wherein the means comprise reagents for performing a diagnostic assay selected from reverse transcription-polymerase chain reaction (RT-PCR) and in situ hybridization (ISH), optionally fluorescence in situ hybridization (FISH).

24. The patient care kit of claim 21, wherein the sample of cancer tissue is a surgical sample, or other biopsy sample obtained from the subject, optionally a biopsy of bone tissue of soft tissue.

25. The patient care kit of claim 21, wherein the Ewing tumor is selected from Ewing tumor of bone (ETB or Ewing sarcoma of bone), extraosseous Ewing tumors (EOE tumors), primitive neuroectodermal tumors (PNET or peripheral neuroepithelioma), and Askin tumors (PNET of the chest wall), optionally a bone sarcoma or a soft tissue sarcoma, optionally, in the legs, pelvis, and/or chest wall of the subject.

26-39. (canceled)
